(19) United States
(12) Patent Application Publication
(10) Pub. No.: US 2015/0183721 A1
(43) Pub. Date: Jul. 2, 2015

(54) AMINO-ALCOHOL ANALOGUES AND USES THEREOF

(71) Applicant: YISSUM RESEARCH DEVELOPMENT COMPANY OF THE HEBREW UNIVERSITY OF JERUSALEM,, JERUSALEM (IL)

(72) Inventors: Arie DAGAN, Jerusalem (IL); Claudia M. BARZILAY, Reut (IL); Amona A. ALI, Kfar Bina (IL)

(21) Appl. No.: 14/659,898

(22) Filed: Mar. 17, 2015

Related U.S. Application Data
(63) Continuation-in-part of application No. 13/388,553, filed on Feb. 2, 2012, now abandoned.
(60) Provisional application No. 61/231,083, filed on Aug. 4, 2009.

(51) Int. Cl. C07C 215/08

(52) U.S. Cl. CPC C07C 215/08

(57) ABSTRACT

This invention relates to amino-alcohol analogues and uses thereof in the treatment of diseases and disorders such as cancer, neurodegenerative and metabolic diseases and genetic storage diseases.

### FIELD OF THE INVENTION

This invention relates to amino-alcohol analogues and uses thereof in the treatment of diseases and disorders such as cancer, neurodegenerative and metabolic diseases and genetic storage diseases.

### BACKGROUND OF THE INVENTION

In the past decade, a substantial progress has been made in the understanding of how sphingolipids contribute to disease-associated processes, leading to novel therapeutic approaches based on interventions in sphingolipid homeostasis. Some of the areas in which particularly important advances have been made are cancer, lipid storage diseases, immunity, inflammation, cystic fibrosis, emphysema, diabetes, sepsis, cardiovascular and neurological diseases.

The attenuation of ceramide levels and/or elevation of S1P are implicated in various stages of cancer pathogenesis, including an anti-apoptotic phenotype, metastasis and escape from senescence Inhibition of the metabolic pathways of these sphingolipids is considered to lead to ceramide accumulation and/or S1P reduction, both serving as targets for anticancer therapy. Therefore, many sphingolipid analogues have been developed. But, so far, none of these have been approved for clinical use.

The synthesis and use of certain lipidic amino-alcohol compounds was presented in the Israel Chemical Society, 2008 [1].

### REFERENCES

[1] 73^{rd} Annual Meeting of the Israel Chemical Society, 4 Feb. 2008, Jerusalem, Israel [http://www.congress.co.il/chemistry08/images/pdf/abstracts.pdf] page 203.
[2] Bhor, S.; Mehltretter, G.; Dobler, C.; Fischer, C.; Beller, M. K., M., A simple and convenient method for epoxidation of olefins without metal catalysts. Advanced Synthesis & Catalysis 2003, 345, (3), 389-392.
[3] Auge, J.; Leroy, F., Lithium trifluoromethanesulfonate-catalysed aminolysis of oxiranes. Tetrahedron Letters 1996, 37, (43), 7715-7716.
[4] Dagan, A.; Wang, C. B.; Fibach, E.; Gott, S., Synthetic, non-natural sphingolipid analogs inhibit the biosynthesis of cellular sphingolipids, elevate ceramide and induce apoptotic cell death. Biochimica Et Biophysica Acta-Molecular and Cell Biology of Lipids 2003, 1633, (3), 161-169.
[5] J. Am. Chem. Soc. 2004, 126, 3686-3687

### SUMMARY OF THE INVENTION

The inventors of the invention disclosed herein have developed novel non-natural sphingolipid analogues which have shown better therapeutic activity, particularly anticancer activity, in comparison to cis-Pt, in various cancer cell-lines such as colon, lung and ovarian cancer cell-lines. A structure activity relationship (SAR) study was performed in order to understand the importance of the lipophilic groups of the synthetic compounds. Systematic changes of lipophilicity in two different sites of the synthetic molecules were studied and have been used to establish the uniqueness of the compounds of the invention. Fluorescent procedures which have been utilized for studying the inhibition of enzymes of sphingolipid metabolism, provided insight into the possible mechanisms of ceramide accumulation resulting in apoptotic death of cancer cells.

In one aspect of the present invention there is provided an amino-alcohol compound of the general formula (I), or a salt or isomer thereof:

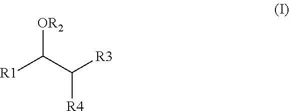

wherein

R₁ is selected from C₈-C₁₄ alkyl, C₁₆-C₂₄ alkyl, C₂-C₂₄ alkenyl, and C₂-C₂₄ alkynyl, each being optionally substituted with at least one substituent selected from —OH, —OR₅ and optionally substituted C₆-C₁₀ aryl;

R₂ is selected from —H, C₁-C₆ alkyl, C₂-C₆ alkenyl, C₂-C₆ alkynyl, and —C(═O)—R₆;

R₃ is selected from —H, C₁-C₂₄ alkyl, C₂-C₂₄ alkenyl, and C₂-C₂₄ alkynyl, each being optionally substituted with at least one substituent selected from —OH, —OR₇ and optionally substituted C₆-C₁₀ aryl;

R₄ is selected from —NHR₈, —NR₈R₉ and —N⁺R₈R₉R₁₀;

each of R₅ and R₇, independently of each other is selected from C₁-C₆ alkyl, C₂-C₆ alkenyl, C₂-C₆ alkynyl, and —C(═O)—R₁₁;

each of R₆ and R₁₁, independently of each other is selected from C₁-C₆ alkyl, C₂-C₆ alkenyl, and C₂-C₆ alkynyl;

each of R₈ and R₉, independently of the other, is selected from C₁-C₂₄ alkyl, C₂-C₂₄ alkenyl, C₂-C₂₄ alkynyl, —C(═S)—R₁₂, —C(═S)—NR₁₂R₁₃, —SO₂—R₁₂, —C(═O)—R₁₂, and —C(═O)—NR₁₂R₁₃;

R₁₀ is selected from —H, C₁-C₂₄ alkyl, C₂-C₂₄ alkenyl, C₂-C₂₄ alkynyl, —C(═S)—R₁₂, —C(═S)—NR₁₂R₁₃, —SO₂—R₁₂, —C(═O)—R₁₂, and —C(═O)—NR₁₂R₁₃;

when R₄ is —NR₈R₉, R₈ and R₉, together with the N atom to which they are bonded may form a heterocyclic group, optionally comprising one or more additional atom selected from N, S, and O;

each of R₁₂ and R₁₃, independently of each other is selected from C₁-C₆ alkyl, C₂-C₆ alkenyl and C₂-C₆ alkynyl; and

wherein at least one of R₁ and R₈ is selected from C₉-C₂₄ alkyl, C₉-C₂₄ alkenyl and C₉-C₂₄ alkynyl.

In compounds of the invention, R₁ is different from a linear C₁₅ alkyl.

In some embodiments, depending on the nature of R₄, particularly R₈, R₁ may be a linear C₁₅ alkyl.

As used herein, the term “alkyl” refers to branched or linear carbon chain of sp³ hybridized carbon atoms, with each carbon atom being bonded to a neighboring carbon atom through a single C—C bond. The alkyl may be optionally substituted with one or more substituents, being all the same or different, or of any combination. The substitution may be a mid-chain substitution, namely not at a terminal carbon but rather on any other carbon of the alkyl chain, or at the terminal carbon. Each carbon of the alkyl chain may be optionally substituted with one or two substituents. The terminal carbon may be substituted with one, two or three substituents.

The expression “C₈-C₁₄ alkyl” refers to an alkyl chain having between 8 and 14 carbon atoms, with any sub-rang being within the scope of this expression. For example, C₈-C₁₄ alkyl also includes such alkyls as having from 8 to 13 carbon atoms, from 8 to 12, from 8 to 11, from 8 to 10, from 8 to 9 and from 9 to 14, from 9 to 13, from 9 to 12 . . . , etc., as well as alkyls of a specific number of carbon atoms within that range: 8, 9, 10, 11, 12, 13 and 14.

Exemplary alkyl groups herein include, but are not limited to octyl, nonyl, decyl, undecyl, dodecyl and others.

Similarly, the term “alkenyl” and “alkynyl refer to carbon chains having at least one double bond or at least one triple bond, respectively. A “C₂-C₂₄ alkenyl”, similarly to the above, is a carbon chain, linear or branched, and optionally substituted, having between 2 and 24 carbon atoms of which at least two carbon atoms form a C—C double bond. A “C₂-C₂₄ alkynyl” similarly refers to a carbon chain, linear or branched and optionally substituted, having between 2 and 24 carbon atoms, of which at least two carbon atoms form a C—C triple bond.

The double or triple bond may be a mid-chain bond (namely any bond other than a bond to the terminal carbon atom) or a terminal chain (end-chain) bond. Each alkenyl or alkynyl may have multiple bonds, one or more of which may be a terminal bond and the remaining may be mid-chain bonds. Where multiple double and/or triple bonds are present, they may or may not be at alternating bonds. The double bonds may be either cis or trans.

As used herein, “aryl” refers to an aromatic monocyclic or multicyclic groups containing from 6 to 10 carbon atoms. Aryl groups include, but are not limited to groups such as unsubstituted or substituted phenyl and unsubstituted or substituted naphthyl. Where applicable, the aryl moiety may be substituted with one or more substituents. Thus, the expression “optionally substituted C₆-C₁₀ aryl” refers to an aryl, as defined, in having one or more substituents selected from C₁-C₆ alkyl, C₂-C₆ alkenyl, C₂-C₆ alkynyl, —OH, —O—C₁-C₆ alkyl and a halide (I, Br, Cl, F). Where one substituent is present, it may be at the ortho-, meta- or para-position to the ipso carbon. Where two or more substituents are present, each substituting group may be at any position to each other or relative to the ipso carbon. For example, where two substituents are present, the substitution on the aryl group, taking into account the ipso carbon, may be 1,2,3; 1,3,4; 1,4,5; 1,5,6; 1,2,4; 1,2,5; 1,2,6; 1,3,5; 1,3,6, etc. Each of said two or more substituents may be the same or different.

The designation “—C(═O)—R₆”, for example with reference to variant R₂, refers to a carbon substituent having one bond to the oxygen atom to which R₂ is bonded, a single bond to R₆, as defined, and a double bond to an oxygen atom.

As defined for a compound of general formula (I), R₄ may be selected from —NHR₈, —NR₈R₉ and —N⁺R₈R₉R₁₀. Each of R₈, R₉ and R₁₀, may be each selected so that all three variants are the same, different or a combination thereof (i.e., two may be the same and the third different). In some embodiments, when R₄ is —NHR₈ or —NR₈R₉, R₈ and R₉ are not —H, namely R₄ is different from —NH₂. Similarly, where R₄ is —N⁺R₈R₉R₁₀, the variants R₈, R₉ and R₁₀ are different from —H, namely R₄ is not —N⁺H₃.

When R₄ is —NR₈R₉, R₈ and R₉, together with the N atom to which they are bonded may form a heterocyclic group, optionally comprising one or more additional atom selected from N, S, and O. The heterocyclic group which is formed, may have a 5- 6- or 7-membered heterocyclic ring comprising one or more additional heteroatom selected from N, S and O. Non-limiting examples of such heterocyclic ring systems are pyrrolidinyl, 2- or 3-pyrrolinyl, imidazolyl, pyrazolyl, imidazolidinyl, oxazolidinyl, thiazolidinyl, 1,2,3-triazolinyl, 1,2,4-triazolinyl, pyridinyl, piperidinyl, piperazinyl, oxazinyl, azepinyl, diazepinyl and others.

In some embodiments, in a compound of general formula (I), R₁ is selected from unsubstituted C₈-C₁₄ alkyl, C₁₆-C₂₄ alkyl, C₂-C₂₄ alkenyl, and C₂-C₂₄ alkynyl.

In some embodiments, R₁ is C₈-C₁₄ alkyl, being selected, in different embodiments, from C₈-C₁₄ alkyl, C₉-C₁₄ alkyl, C₁₀-C₁₄ alkyl, C₁₁-C₁₄ alkyl, C₁₂-C₁₄ alkyl, C₁₃-C₁₄ alkyl, C₈-C₁₃ alkyl, C₉-C₁₃ alkyl, C₁₀-C₁₃ alkyl, C₁₁-C₁₃ alkyl, C₁₂-C₁₃ alkyl, C₈-C₁₄ alkyl, C₉-C₁₄ alkyl, C₁₀-C₁₄ alkyl, C₁₁-C₁₄ alkyl, C₁₂-C₁₄ alkyl, C₁₃-C₁₄ alkyl, C₈-C₁₃ alkyl, C₉-C₁₃ alkyl, C₁₀-C₁₃ alkyl, C₁₁-C₁₃ alkyl, C₁₂-C₁₃ alkyl, C₈-C₁₂ alkyl, C₉-C₁₂ alkyl, C₁₀-C₁₂ alkyl, C₁₁-C₁₂ alkyl, C₈-C₁₁ alkyl, C₉-C₁₁ alkyl, C₁₀-C₁₁ alkyl, C₈-C₁₀ alkyl, C₉-C₁₀ alkyl and C₈-C₉ alkyl.

In some embodiments, R₁ is an alkyl having 9, 10, 11, 12, 13, 14, 16, 17, 18, 19, 20, 21, 22, 23 or 24 carbon atoms in a continuous aliphatic chain (which may or may not be branched or further substituted). Thus, the compound of formula (I) is a compound of formula (II):

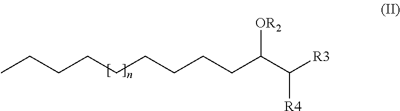

wherein

n is an integer selected from 1, 2, 3, 4, 5, 6, 8, 9, 10, 11, 12, 13, 14, 15 and 16, and each of R₂, R₃ and R₄ are as defined above.

In a compound of formula (II), in some embodiments, n is 1 or 8.

In other embodiments, in a compound of the invention, R₂ is —H or C₁-C₆ alkyl.

In other embodiments, R₃ is selected from —H and C₁-C₂₄ alkyl.

In further embodiments, in a compound of the invention, R₄ is —NHR₈ and R₈ is not —H, wherein the N atom is optionally further protonated or R₄ is —NR₈R₉, and the N atom is optionally further protonated. When R₄ is —NR₈R₉, R₈ may be —H or a group different from —H and R₉ is a group different from —H.

Any compound of the present invention, due to the presence of the N atom of R₄, or any other N atom of a substituent present in the compound, may exist as a salt, where one or more of the N atoms may be in the form of a quaternary amino due to protonation or alkylation. The counter-anion may be an organic or inorganic anion as further detailed hereinbelow.

In some embodiments, R₁ is selected from C₈-C₁₄ alkyl and C₁₆-C₂₄ alkyl, as defined, R₄ is —NHR₈ and the compound is of the general formula (III):

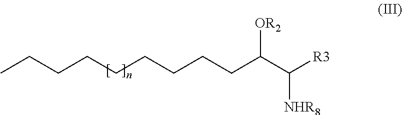

wherein

n, R₂ and R₃ are as defined above, and R₈ is different from —H.

In some embodiments, in a compound of formula (III), R₈ is C₁-C₂₄ alkyl, as defined above.

In further embodiments, R₈ is C₁-C₁₆ alkyl. In other embodiments, R₈ is an alkyl chain having 2, 3, 4, 5, 6, 7, 8, 9, 10, 11, 12, 13, 14, 15, or 16 carbons in a continuous aliphatic chain which may be branched or further substituted as disclosed hereinabove.

In further embodiments, R₈ is —NR₈R₉, the N atom may be further protonated, the compound being a compound of formula (IV):

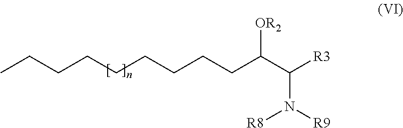

wherein

each of n, R₂, and R₃ are as defined above and R₈ and R₉ are each different from —H.

In some embodiments, each of R₈ and R₉ are different or same —C₁-C₂₄ alkyl. In some embodiments, R₈ and R₉ are the same alkyl group. In other embodiments, R₈ and R₉ are of different alkyl chains, namely, each having a different number of carbon atoms, different chain length, and/or different substitution.

In some embodiments, one of R₈ and R₉ is an alkyl having up to three carbon atoms and the other of R₈ and R₉ is an alkyl having 4 or more carbon atoms.

In some further embodiments, each of R₈ and R₉, independently of each other, is an alkyl chain having 2, 3, 4, 5, 6, 7, 8, 9, 10, 11, 12, 13, 14, 15, or 16 carbons in a continuous aliphatic chain which may be branched or further substituted as disclosed hereinabove.

The N atom bearing R₈ and R₉ may be protonated or substituted by R₁₀ to form —N⁺HR₈R₉ or —N⁺R₈R₉R₁₀, respectively.

In further embodiments, R₁ is C₁-C₂₄ alkyl, R₃ is —H and R₄ is selected from —NHR₈ and —NR₈R₉ and R₂ is —H.

The invention further provides a compound selected amongst compounds herein designated A1, A2, A3, A4, A5, A6, A7, A8, A9, A10, A11 and A12:

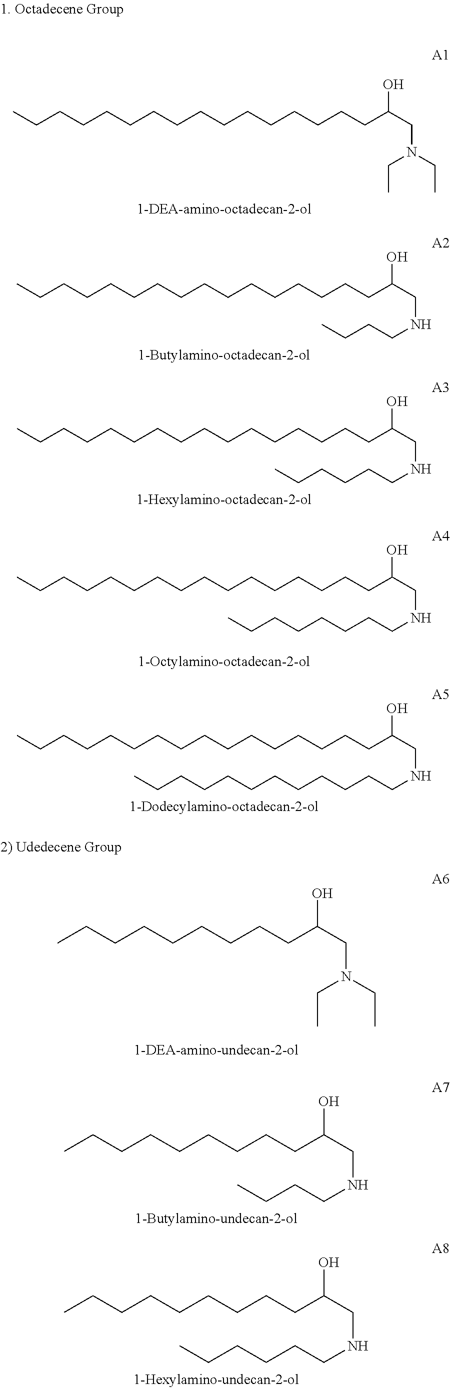

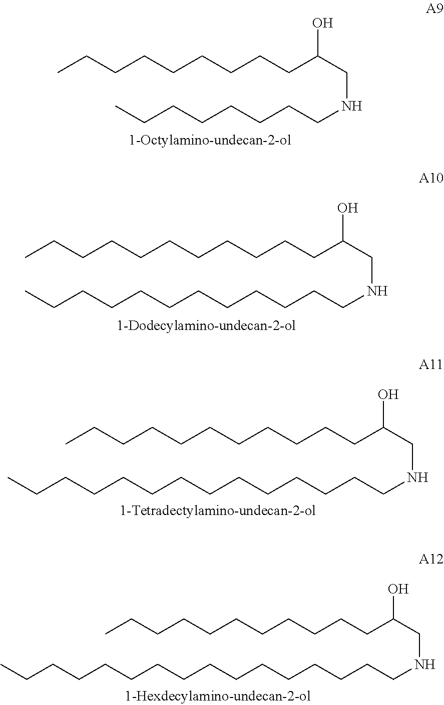 [SMILES: CCCCCCCCCC(O)CNCCCCCCCC ; CCCCCCCCCCCCNCC(O)CCCCCCCCCCC ; CCCCCCCCCCCCCCNCC(O)CCCCCCCCCCC ; CCCCCCCCCCCCCCCCNCC(O)CCCCCCCCCCC]

As a person skilled in the art would appreciate, the compounds of the present invention contain at least two chiral centers. Such chiral centers may be of either the (R) or (S) configuration, or may be a mixture thereof. Thus, the compounds provided herein may be enantiomerically pure, or be stereoisomeric or diastereomeric mixtures. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

The compounds of the invention, additionally, contain at least two acid/base centers, i.e., the oxygen and nitrogen atoms of the amino-alcohol backbone, which may undergo protonation, deprotonation or further alkylation under a variety of conditions. The compounds of the invention may, therefore, exist in one or more salt forms. The salt forms may or may not be pharmaceutically acceptable salts.

Pharmaceutically acceptable salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, ethylene-diamine, N-methylglucamine, and procaine (see, for example, Berge S. M., et al., “Pharmaceutical Salts, ” (1977) J. of Pharmaceutical Science, 66: 1-19). The salts may also be pharmaceutically acceptable quaternary salts, such as a quaternary salt having the structure NR′R″R′″Z, wherein R′, R″ and R′″, each is independently selected from hydrogen, alkyl or benzyl and Z is a counterion, such as a halide, e.g., chloride, bromide, iodide, O-alkyl, toluenesulfonate, methylsulfonate, sulfonate, phosphate, or carboxylate.

Pharmaceutically acceptable acid addition salts of compounds of the invention include salts derived from inorganic acids such as hydrochloric, nitric, phosphoric, sulfuric, hydrobromic, hydriodic, phosphorous, and the like, as well as salts derived from organic acids such as aliphatic mono-and dicarboxylic acids, phenyl-substituted alkanoic acids, hydroxy alkanoic acids, alkanedioic acids, aromatic acids, aliphatic and aromatic sulfonic acids, and others. Such salts include sulfate, pyrosulfate, bisulfate, sulfite, bisulfite, nitrate, phosphate, monohydrogenphosphate, dihydrogenphosphate, metaphosphate, pyro-phosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate or galacturonate (see, for example, Berge S. M., et al., “Pharmaceutical Salts, ” (1977) J. of Pharmaceutical Science, 66: 1-19).

The acid addition salts of compounds of the invention may be prepared by contacting the free base form, through for example the N atom at position R₄, with a sufficient amount of the desired acid or an alkylating agent to produce the salt in the conventional manner In the protonated cases, the free base form may be regenerated by contacting the salt form with a base and isolating the free base in the conventional manner. The free base forms may differ from their respective salt forms somewhat in certain physical properties such as solubility in polar solvents, but otherwise the salts are equivalent to their respective free base for purposes of the present invention.

The base addition salts of said acidic compounds are prepared by contacting the free acid form with a sufficient amount of the desired base to produce the salt in the conventional manner The free acid form may be regenerated by contacting the salt form with an acid and isolating the free acid in the conventional manner The free acid forms may differ from their respective salt forms somewhat in certain physical properties such as solubility in polar solvents, but otherwise the salts are equivalent to their respective free acid for purposes of the present invention.

The compounds of the invention may be prepared by a variety of synthetic methodologies as known to a person versed in the art of organic synthesis. The invention provides, in another of its aspects, one such method for the preparation of compounds of the invention, a method based on the employment of an epoxide or an aziridine precursor in ring-opening reactions with no emphasis on the stereochemistry of the products. Thus, the products may be pure isomers or any mixture thereof. As a person skilled in the art would appreciate, the stereochemistry of the end products may be controlled and compounds of specific stereochemistry may be prepared and isolated.

Thus, the method for the preparation of compounds of the invention comprises contacting an epoxide or an aziridine precursor molecule with a nitrogen or oxygen nucleophile, respectively, under conditions permitting ring opening of said epoxide or aziridine precursor. Next, functionalization of the N atom or the O atom (or any other atom of the molecule) may take place.

In some embodiments, the precursor is an epoxide containing compound and the nucleophile is a nitrogen nucleophile, such as an alkylamine.

In other embodiments, the precursor is an aziridine containing molecule and the nucleophile is an oxygen nucleophile, such as an alkyloxy.

The compounds of the invention may be used for the preparation of compositions or formulations for a great variety of applications. Thus, the invention also provides use of at least one compound according to the invention, as herein disclosed, for the preparation of a composition. In some embodiments, the composition is a pharmaceutical composition.

Where pharmaceutical applications are contemplated, the compositions of the invention may include at least one compound of the invention, alone or in combination with at least one other drug or agent known in the art. The at least one other drug or agent may be suitable for the treatment or prophylaxis of the same disease or disorder as the compound of the invention, or may be used in combination to modulate (enhance or reduce) at least one effect (therapeutic or otherwise toxic) associated with the use of the compound of the invention.

The pharmaceutical compositions of the invention may or may not include also a carrier. Suitable pharmaceutically acceptable carriers, for example, vehicles, adjuvants, excipients, or diluents, are well-known to those who are skilled in the art and are readily available to the public. Typically, the pharmaceutically acceptable carrier is one which is chemically inert to the active compound (e.g., the compound of the invention and/or at least one additional ingredient, if present) and one which has no detrimental side effects or toxicity under the conditions of use.

The choice of a carrier will be determined in part by the particular compound (e.g., its physical or chemical characteristics), as well as by the particular method used to administer the composition. Accordingly, there is a wide variety of suitable formulations of the pharmaceutical composition of the present invention. The following formulations for oral, aerosol, parenteral, subcutaneous, intravenous, intramuscular and interperitoneal administration are merely exemplary and are in no way limiting.

Formulations suitable for oral administration may comprise of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms may be of the ordinary hard- or soft-shelled gelatin type containing, for example, a surfactant, a lubricant, and inert filler, such as lactose, sucrose, calcium phosphate, and corn starch. Tablet forms may include one or more of lactose, sucrose, mannitol, corn starch, potato starch, alginic acid, microcrystalline cellulose, acacia, gelatin, guar gum, colloidal silicon dioxide, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The compounds of the present invention, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. A compound of the invention or a mixture of two or more compounds may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which may be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxy-ethylenepolypropylene copolymers, (d) amphoteric detergents such as, for example, alkyl-β-aminopriopionates, and 2-alkyl-imidazoline quaternary ammonium salts, and (3) mixtures thereof.

In some embodiments, the parenteral formulations employed for the treatment or prophylaxis of certain diseases or disorders may contain from about 0.5 to about 25% by weight of the compound of the invention in solution. Suitable preservatives and buffers may be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations may range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations may be presented in unit-dose or multi-dose sealed containers, such as ampules and vials, and may be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds of the present invention may also be made into injectable formulations. The requirements for effective pharmaceutical carriers for injectable compositions are well known to those of ordinary skill in the art. See Pharmaceutics and Pharmacy Practice, J.B. Lippincott Co., Philadelphia, Pa., Banker and Chalmers, eds., pages 238-250 (1982), and ASHP Handbook on Injectable Drugs, Toissel, 4^{th} ed., pages 622-630 (1986).

The compounds and pharmaceutical compositions of the invention are effective in the treatment and prophylaxis of a Lipid Storage Disease. Thus, also contemplated is the use of a compound or composition of the invention in a method for treating a disease or a disorder such as Niemann-Pick.

The compounds and pharmaceutical compositions of the invention are effective in the treatment and prophylaxis of cancer. Thus, also contemplated is the use of a compound or composition of the invention in a method for treating a disease or a disorder.

In an additional aspect, the invention generally provides a method for modulating (e.g., increasing, decreasing or maintaining level of) ceramide accumulation in a subject, said method comprising administering to a subject in need of such treatment at least one compound or pharmaceutical composition according to the invention.

In some embodiments, the subject is suffering or has predisposition to suffering from a disease or disorder which is induced by an increase or a decrease in ceramide accumulation in the subject.

In some embodiments, the disease or disorder is cancer.

Also provided is a method of inducing apoptosis of cancer cells (in vivo or in vitro), said method comprising contacting a cancer cell with at least one compound or composition according to the present invention.

In some embodiments, the cancer cell is in the subject's body (human or non-human).

As used herein, “cancer” refers to any carcinoma, any sarcoma, liquid tumors (e.g., multiple myeloma, Waldenstroms' (IgM) gammopathy, Bergers (IgA), CNS lymphoma (e.g., associated with AIDS), gonadal lymphomas and leukemias, mantle cell lymphomas, vascularized stages of leukemias (bone marrow) and lymphomas (in the lymph nodes), and any other leukemia or lymphoma, including low grade leukemias and lymphomas), solid tumors (i.e., vascularized tumors, including angiosarcomas, Kaposi's sarcoma, mesothelioma, Ewing's Sarcoma, choriocarcinoma, ascitis tumors such as ovarian cancer especially with peritoneal implants), gonadal cancers (including ovarial and cervical), airway cancers (small cell lung, lung, and bronchial), gastrointestinal cancers (pancreatic, intestinal, colon, rectal, small intestinal, polyposis, gall duct, stomach), esophageal cancer, Barrett's esophagus cancer, oral cancer, parotid cancer, nasopharyngeal cancer, thyroid cancer, CNS cancers (glial, neuroblastoma multiforme, neuromas, meningiomas, astrocytomas, any other pediatric or adult CNS cancer), urogenital cancers (bladder, renal, adrenal, prostate), skin cancers (melanoma nodular, invasive, superficial spreading MF, squamous cell, lip) bone and connective tissue cancers (breast, bone, chondromas, leiomyomas, Wilm's tumor, retinoblastoma), and any other solid carcinoma or sarcoma of pediatric or adult patients.

In some embodiments, the cancer to be treated by the methods and compositions of the invention is selected from lung cancer, non small cell lung (NSCL) cancer, bronchioalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers.

In further embodiments, the cancer is selected from colon, lung, breast, pancreas and ovarian cancers.

In additional embodiments, the cancer is selected from colon, lung and ovarian cancers.

The invention also provides a method for the treatment of a disease or disorder, said method comprising administering to a subject suffering from said disease or disorder an effective amount of at least one compound or composition according to the invention.

In some embodiments, said disease or disorder is selected amongst cancer, neurodegenerative and metabolic diseases and genetic storage diseases.

In some embodiments, said disease or disorder is cancer.

The methods of the invention may be used in patients who are treatment naive, in patients who have previously received or are currently receiving treatment with other pharmaceutical agents or combinations, e.g., anti-cancer agents. Other subjects may include patients that have metastasis or no metastasis.

In some embodiments, the treatment with said at least one compound or composition of the invention precedes, follows or in combination with existing therapeutic modalities, which may or may not involve the administration of one or more agent selected from a chemoagent, an immunotherapeutic, a cancer vaccine, an anti-angiogenic agent, a cytokine, hormone therapy, gene therapy, a biological therapy and radiotherapy.

The “effective amount” for purposes herein is determined by such considerations as may be known in the art. The amount should be effective to achieve the desired therapeutic effect as described herein, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half life in the body, on undesired side effects, if any, on factors such as age and gender, etc

The term “treatment and/or prophylaxis”, or any lingual variation thereof, as used herein refers to the administering of a therapeutic amount of a compound or a composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

In some embodiments there is provided a compound of the general formula (I), or a salt or isomer thereof:

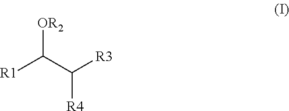

wherein

R₁ is selected from C₈-C₁₄ alkyl, C₁₆-C₂₄ alkyl, C₂-C₂₄ alkenyl, and C₂-C₂₄ alkynyl, each being optionally substituted with at least one substituent selected from —OH, —OR₅ and optionally substituted C₆-C₁₀ aryl;

R₂ is selected from —H, C₁-C₆ alkyl, C₂-C₆ alkenyl, C₂-C₆ alkynyl, and —C(═O)—R₆;

R₃ is selected from —H, C₁-C₂₄ alkyl, C₂-C₂₄ alkenyl, and C₂-C₂₄ alkynyl, each being optionally substituted with at least one substituent selected from —OH, —OR₇ and optionally substituted C₆-C₁₀ aryl;

R₄ is selected from —NHR₈, —NR₈R₉ and —N⁺R₈R₉R₁₀;

each of R₅ and R₇, independently of each other is selected from C₁-C₆ alkyl, C₂-C₆ alkenyl, C₂-C₆ alkynyl, and —C(═O)—R₁₁;

each of R₆ and R₁₁, independently of each other is selected from C₁-C₆ alkyl, C₂-C₆ alkenyl, and C₂-C₆ alkynyl;

each of R₈ and R₉, independently of the other, is selected from C₁-C₂₄ alkyl, C₂-C₂₄ alkenyl, C₂-C₂₄ alkynyl, —C(═S)—R₁₂, —C(═S)—NR₁₂R₁₃, —SO₂—R₁₂, —C(═O)—R₁₂, and —C(═O)—NR₁₂R₁₃;

R₁₀ is selected from —H, C₁-C₂₄ alkyl, C₂-C₂₄ alkenyl, C₂-C₂₄ alkynyl, —C(═S)—R₁₂, —C(═S)—NR₁₂R₁₃, —SO₂—R₁₂, —C(═O)—R₁₂, and —C(═O)—NR₁₂R₁₃;

when R₄ is —NR₈R₉, R₈ and R₉, together with the N atom to which they are bonded may form a heterocyclic group, optionally comprising one or more additional atom selected from N, S, and O;

each of R₁₂ and R₁₃, independently of each other is selected from C₁-C₆ alkyl, C₂-C₆ alkenyl and C₂-C₆ alkynyl; and

wherein at least one of R₁ and R₈ is selected from C₉-C₂₄ alkyl, C₉-C₂₄ alkenyl and C₉-C₂₄ alkynyl.

In some embodiments, R₁ is selected from unsubstituted C₈-C₁₄ alkyl, and C₁₆-C₂₄ alkyl.

In some embodiments, R₁ is an alkyl having 9, 10, 11, 12, 13, 14, 16, 17, 18, 19, 20, 21, 22, 23 or 24 carbon atoms in a continuous aliphatic chain.

In some embodiments, the compound is a compound of formula (II):

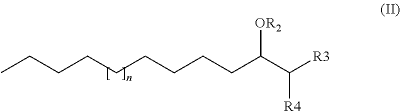

wherein

n is an integer selected from 1, 2, 3, 4, 5, 6, 8, 9, 10, 11, 12, 13, 14, 15, and 16, and each of R₂, R₃ and R are as defined herein.

In some embodiments, n is 1 or 8.

In some embodiments, R₂ is —H or C₁-C₆ alkyl.

In some embodiments, R₃ is selected from —H ad C₁-C₂₄ alkyl.

In some embodiments, R₄ is —NHR₈ and R₈ is not —H.

In some embodiments, R₄ is —NR₈R₉ and wherein R₈ is —H or a group different from —H and R₉ is a group different from —H.

In some embodiments, R₁ is selected from C₈-C₁₄ and C₁₆-C₂₄ alkyl, and R₄ is —NHR₈.

In some embodiments, the compound is of the general formula (III):

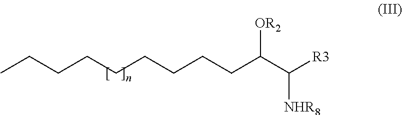

wherein

n, R₂ and R₃ are as defined and R₈ is different from —H.

In some embodiments, R₈ is C₁-C₂₄ alkyl.

In some embodiments, R₈ is C₁-C₁₆ alkyl.

In some embodiments, R₄ is —NR₈R₉, the compound being of formula (IV):

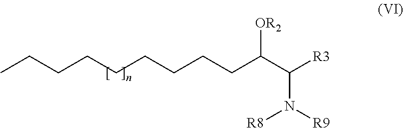

wherein

each of n, R₂ and R₃ are as defined in claim 1 and R₈ and R₉ are each different from —H.

In some embodiments, each of R₈ and R₉ is different or same —C₁-C₂₄ alkyl.

In some embodiments, R₈ and R₉ are the same —C₁-C₂₄ alkyl.

In some embodiments, each of R₈ and R₉ is an alkyl chain having 2, 3, 4, 5, 6, 7, 8, 9, 10, 11, 12, 13, 14, 15, or 16 carbon atoms in a continuous aliphatic chain.

In some embodiments, there is provided a compound selected from compounds herein designated A1, A2, A3, A4, A5, A6, A7, A8, A9, A10, A11 and A12.

In some embodiments, the pharmaceutical composition comprising a compound of the general formula (I), or a salt or isomer thereof:

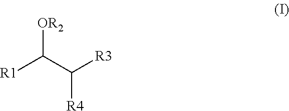

wherein

R₁ an alkyl having 9, 10, 11, 12, 13, 14, 16, 17, 18, 19, 20, 21, 22, 23 or 24 carbon atoms in a continuous unsubstituted aliphatic chain;

R₂ is selected from —H, C₁-C₆ alkyl, C₂-C₆ alkenyl, C₂-C₆ alkynyl, and —C(═O)—R₆;

R₃ is selected from —H, C₁-C₂₄ alkyl, C₂-C₂₄ alkenyl, and C₂-C₂₄ alkynyl, each being optionally substituted with at least one substituent selected from —OH, —OR₇ and optionally substituted C₆-C₁₀ aryl;

R₄ is selected from —NHR₈ and —NR₈R₉;

R₇ is selected from C₁-C₆ alkyl, C₂-C₆ alkenyl, C₂-C₆ alkynyl, and —C(═O)—R₁₁;

each of R₆ and R₁₁, independently of each other is selected from C₁-C₆ alkyl, C₂-C₆ alkenyl, and C₂-C₆ alkynyl;

where R₄ is —NHR₈, R₈ is an alkyl chain having 5, 6, 7, 9, 10, 11, 12, 13, 14, 15, or 16 carbon atoms in a continuous unsubstituted linear aliphatic chain;

where R₄ is —NR₈R₉, each of R₈ and R₉, independently of each other, is an alkyl chain having 4, 5, 6, 7, 8, 9, 10, 11, 12, 13, 14, 15, or 16 carbon atoms in a continuous unsubstituted linear aliphatic chain.

In some embodiments, R₂ is —H or C₁-C₆ alkyl.

In some embodiments, R₃ is selected from —H and C₁-C₂₄ alkyl.

In some embodiments, each of R₂ and R₃ is selected from —H.

In some embodiments, R₄ is —NHR₈.

In some embodiments, R₄ is —NR₈R₉ and R₈ is —H or a group different from —H and R₉ is a group different from —H.

In some embodiments, R₁ is an alkyl having 9, 10, 11, 12, 13, 14, 16, 17, 18, 19, 20, 21, 22, 23 or 24 carbon atoms in a continuous unsubstituted aliphatic chain, and R₄ is —NHR₈.

In some embodiments, there is provided a method for the treatment or prophylaxis of a disease or disorder, said method comprising administering to a subject suffering from said disease or disorder an effective amount of the pharmaceutical composition according to the invention.

In some embodiments, said disease or disorder is cancer.

In some embodiments, said disease or disorder is ovarian carcinoma, lung carcinoma, or colon carcinoma.

In some embodiments, the compound is of formula (II):

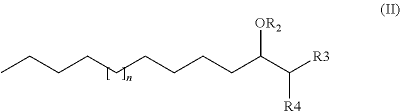

wherein

n is an integer selected from 1, 2, 3, 4, 5, 6, 8, 9, 10, 11, 12, 13, 14, 15, and 16,

R₂ is selected from —H, C₁-C₆ alkyl, C₂-C₆ alkenyl, C₂-C₆ alkynyl and —C(═O)—R₆;

R₃ is selected from —H, C₁-C₂₄ alkyl, C₂-C₂₄ alkenyl, and C₂-C₂₄ alkynyl, each being optionally substituted with at least one substituent selected from —OH, —OR₇ and optionally substituted C₆-C₁₀ aryl;

R₆ and R₁₁, independently of each other is selected from C₁-C₆ alkyl, C₂-C₆ alkenyl, and C₂-C₆ alkynyl;

R₇ is selected from C₁-C₆ alkyl, C₂-C₆ alkenyl, C₂-C₆ alkynyl, and —C(═O)—R₁₁;

R₄ is —NHR₈;

R₈ is an alkyl having 12 carbons in a continuous aliphatic chain.

In some embodiments, n is 1, 3 or 8.

In some embodiments, R₂ is —H or C₁-C₆ alkyl.

In some embodiments, R₃ is selected from —H ad C₁-C₂₄ alkyl.

In some embodiments, each of R₂ and R₃ is selected from —H.

In some embodiments, R₄ is —NHR₈.

In some embodiments, R₄ is —NR₈R₉ and R₈ is —H or a group different from —H and R₉ is a group different from —H.

In some embodiments, R₁ is an alkyl having 9, 10, 11, 12, 13, 14, 16, 17, 18, 19, 20, 21, 22, 23 or 24 carbon atoms in a continuous unsubstituted aliphatic chain and R₄ is —NHR₈.

In some embodiments, the compound is of the general formula (III):

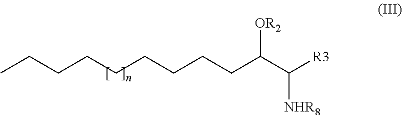

wherein

n is an integer selected from 1, 2, 3, 4, 5, 6, 8, 9, 10, 11, 12, 13, 14, 15, and 16, R₂, R₃ and R₈ are as defined.

In some embodiments, R₄ is —NR₈R₉, the compound being of formula (IV):

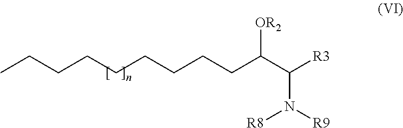

wherein

n is an integer selected from 1, 2, 3, 4, 5, 6, 8, 9, 10, 11, 12, 13, 14, 15, and 16, R₂ and R₃, R₈ and R₉ are as defined.

In some embodiments, R₈ and R₉ are the same alkyl chain having 4, 5, 6, 7, 8, 9, 10, 11, 12, 13, 14, 15, or 16 carbon atoms in a continuous aliphatic chain.

In some embodiments, each of R₈ and R₉ is an alkyl chain having 4, 5, 6, 7, 8, 9, 10, 11, 12, 13, 14, 15, or 16 carbon atoms in a continuous aliphatic chain.

In some embodiments, the compound is selected from compounds herein designated A3, A4, A5, A6, A7, A8, A10, A11 and A12.

In some embodiments, the compound is of the general formula (I), or a salt or isomer thereof:

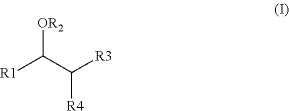

wherein

R₁ is selected from C₈-C₁₄ alkyl, C₁₆-C₂₄ alkyl, C₂-C₂₄ alkenyl, and C₂-C₂₄ alkynyl, each being optionally substituted with at least one substituent selected from —OH, —OR₅ and optionally substituted C₆-C₁₀ aryl;

R₂ is selected from —H, C₁-C₆ alkyl, C₂-C₆ alkenyl, C₂-C₆ alkynyl, and —C(═O)—R₆;

R₃ is selected from —H, C₁-C₂₄ alkyl, C₂-C₂₄ alkenyl, and C₂-C₂₄ alkynyl, each being optionally substituted with at least one substituent selected from —OH, —OR₇ and optionally substituted C₆-C₁₀ aryl;

R₄ is selected from —NHR₈, —NR₈R₉ and —N⁺R₈R₉R₁₀;

each of R₅ and R₇, independently of each other is selected from C₁-C₆ alkyl, C₂-C₆ alkenyl, C₂-C₆ alkynyl, and —C(═O)—R₁₁;

each of R₆ and R₁₁, independently of each other is selected from C₁-C₆ alkyl, C₂-C₆ alkenyl, and C₂-C₆ alkynyl;

each of R₈ and R₉, independently of the other, is selected from C₁-C₂₄ alkyl, C₂-C₂₄ alkenyl, C₂-C₂₄ alkynyl, —C(═S)—R₁₂, —C(═S)—NR₁₂R₁₃, —SO₂—R₁₂, —C(═O)—R₁₂, and —C(═O)—NR₁₂R₁₃;

R₁₀ is selected from —H, C₁-C₂₄ alkyl, C₂-C₂₄ alkenyl, C₂-C₂₄ alkynyl, —C(═S)—R₁₂; —C(═S)—NR₁₂R₁₃, —SO₂—R₁₂; —C(═O)—R₁₂, and —C(═O)—NR₁₂R₁₃;

when R₄ is —NR₈R₉, R₈ and R₉, together with the N atom to which they are bonded may form a heterocyclic group, optionally comprising one or more additional atom selected from N, S, and O;

each of R₁₂ and R₁₃, independently of each other is selected from C₁-C₆ alkyl, C₂-₆ alkenyl and C₂-C₆ alkynyl; and

wherein at least one of R₁ and R₈ is selected from C₉-C₂₄ alkyl, C₉-C₂₄ alkenyl and C₉-C₂₄ alkynyl.

In some embodiments, the compound is of the general formula (I), or a salt or isomer thereof:

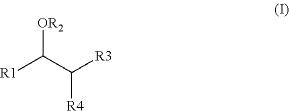

wherein

R₁ is selected from the group consisting of linear unsubstituted C₈-C₁₄ alkyl, linear unsubstituted C₁₆-C₂₄ alkyl, C₂-C₂₄ alkenyl, and C₂-C₂₄ alkynyl, wherein the C₂-C₂₄ alkenyl and C₂-C₂₄ alkynyl being optionally substituted with at least one substituent selected from the group consisting of —OH, —OR₅ and optionally substituted C₆-C₁₀ aryl;

R₂ is selected from —H, C₁-C₆ alkyl, C₂-C₆ alkenyl, C₂-C₆ alkynyl, and —C(═O)—R₆;

R₃ is selected from —H, C₁-C₂₄ alkyl, C₂-C₂₄ alkenyl, and C₂-C₂₄ alkynyl, each being optionally substituted with at least one substituent selected from —OH, —OR₇ and optionally substituted C₆-C₁₀ aryl;

R₄ is selected from —NHR₈, —NR₈R₉;

each of R₅ and R₇, independently of each other is selected from C₁-C₆ alkyl, C₂-C₆ alkenyl, C₂-C₆ alkynyl, and —C(═O)—R₁₁;

each of R₆ and R₁₁, independently of each other is selected from C₁-C₆ alkyl, C₂-C₆ alkenyl, and C₂-C₆ alkynyl;

wherein R₄ is —NHR₈, R₈ is 3, 5, 6, 7, 9, 10, 11, 12, 13, 14, 15, 16, 17, 18, 19, 20, 21, 22, 23, or 24 carbon atoms in a continuous unsubstituted linear aliphatic chain;

wherein R₄ is —NR₈R₉, each of R₈ and R₉, independently of the other is 2, 3, 4, 5, 6, 7, 8, 9, 10, 11, 12, 13, 14, 15, 16, 17, 18, 19, 20, 21, 22, 23, or 24 carbon atoms in a continuous unsubstituted linear aliphatic chain;

and

wherein at least one of R₁ and R₈ is selected from the group consisting of linear unsubstituted C₉-C₂₄ alkyl, C₉-C₂₄ alkenyl and C₉-C₂₄ alkynyl.

In some embodiments, R₁ is selected from the group consisting of linear unsubstituted C₈-C₁₄ alkyl, and linear unsubstituted C₁₆-C₂₄ alkyl.

In some embodiments, R₁ is an alkyl having 9, 10, 11, 12, 13, 14, 16, 17, 18, 19, 20, 21, 22, 23 or 24 carbon atoms in a continuous aliphatic chain.

In some embodiments, the compound being a compound of formula (II):

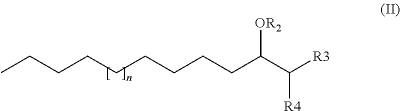

wherein

n is an integer selected from 1, 2, 3, 4, 5, 6, 8, 9, 10, 11, 12, 13, 14, 15, and 16, and each of R₂, R₃ and R are as defined.

In some embodiments, n is 1 or 8.

In some embodiments, R₂ is -H or C₁-C₆ alkyl.

In some embodiments, R₃ is selected from the group consisting of —H and C₁-C₂₄ alkyl.

In some embodiments, R₄ is —NHR₈ and R₈ is not —H.

In some embodiments, R₁ is selected from the group consisting of linear unsubstituted C₈-C₁₄ alkyl and linear unsubstituted C₁₆-C₂₄ alkyl, and R₄ is —NHR₈.

In some embodiments, wherein the compound is of the general formula (III):

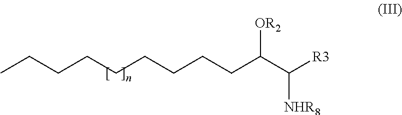

wherein

n, R₂ and R₃ are as defined and R₈ is different from —H.

In some embodiments, R₈ is 3, 5, 6, 7, 9, 10, 11, 12, 13, 14, 15, 16, 17, 18, 19, 20, 21, 22, 23, or 24 carbon atoms in a continuous unsubstituted linear aliphatic chain.

In some embodiments, R₈ is 3, 5, 6, 7, 9, 10, 11, 12, 13, 14, 15, or 16 carbon atoms in a continuous unsubstituted linear aliphatic chain.

In some embodiments, the compound is selected from

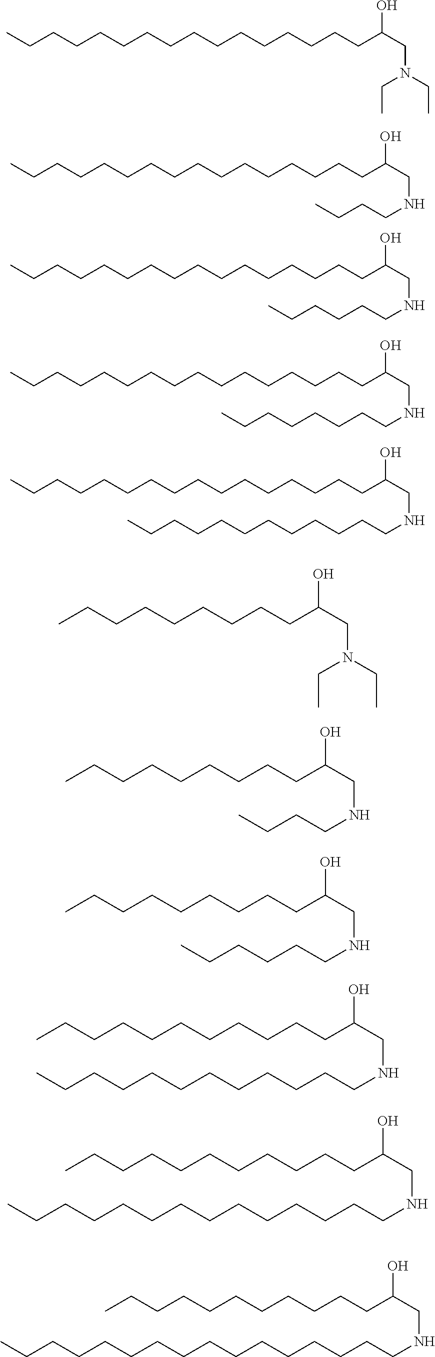

In some embodiments, the compound is selected from:

1-Dodecyl-amino-dodecan-2-ol,

1-Decyl-amino-dodecan-2-ol,

1-DEA-amino-octadecan-2-ol,

1-Butyl-amino-octadecan-2-ol,

1-Hexyl-amino-octadecan-2-ol,

1-Octyl-amino-octadecan-2-ol,

1-Dodecyl-amino-octadecan-2-ol,

1-DEA-amino-undecan-2-ol,

1-Butyl -amino-undecan-2-ol,

1-Hexyl-amino-undecan-2-ol,

1-Dodecyl-amino-tridecan-2-ol,

1-tetradecyl-amino-tridecan-2-ol, and

1-hexadecyl-amino-tridecan-2-ol.

In some embodiments, the compound is selected from:

1-Dodecyl-amino-dodecan-2-ol and,

1-Decyl-amino-dodecan-2-ol.

In some embodiments, the compound is selected from:

1-DEA-amino-octadecan-2-ol,

1-Butyl -amino-octadecan-2-ol,

1-Hexyl-amino-octadecan-2-ol,

1-Octyl-amino-octadecan-2-ol,

1-Dodecyl-amino-octadecan-2-ol,

1-DEA-amino-undecan-2-ol,

1-Butyl -amino-undecan-2-ol,

1-Hexyl-amino-undecan-2-ol,

1-Dodecyl-amino-tridecan-2-ol,

1-tetradecyl-amino-tridecan-2-ol, and

1-hexadecyl-amino-tridecan-2-ol.

In some embodiments, the compound is any one of:

1-DEA-amino-octadecan-2-ol,

1-Butyl-amino-octadecan-2-ol,

1-Hexyl-amino-octadecan-2-ol,

1-Octyl-amino-octadecan-2-ol,

1-Dodecyl-amino-octadecan-2-ol,

1-DEA-amino-undecan-2-ol,

1-Butyl-amino-undecan-2-ol,

1-Hexyl-amino-undecan-2-ol,

1-Dodecyl-amino-tridecan-2-ol,

1-tetradecyl-amino-tridecan-2-ol, and

1-hexadecyl-amino-tridecan-2-ol.

### BRIEF DESCRIPTION OF THE DRAWINGS

In order to understand the invention and to see how it may be carried out in practice, embodiments will now be described, by way of non-limiting example only, with reference to the accompanying drawings, in which:

FIGS. 1A and 2B show the calculated log P versus the IC₅₀ values for: compounds A1-A5 (FIG. 1A octadecene group) and compounds A8-A12 (FIG. 1B undecene group). C log P was calculated by means of EPI Suite V3.11 software.

FIGS. 2A and 2B demonstrate the effect of compounds A2 (FIG. 2A) and A10 (FIG. 2B) on the formation of SPM in A2780 and A2780cisR cancer cell-lines. The nontoxic concentrations that have been used were: 2, 4, 6, 8 μM/well for A2, and 0.25, 0.5, 1, 2 μM/well for A10.

FIGS. 3A and 3B demonstrate the effect of compounds A2 (FIG. 3A) and A10 (FIG. 3B) on the formation of SPM in HT-29 and CRL-5803 cancer cell-lines. The nontoxic concentrations that have been used were: 4, 6, 8, 12 μM/well for A2, and 0.5, 1, 2, 4 μM/well for A10.

FIG. 4 presents the results of Capase-3 assay for apoptosis determination.

### DETAILED DESCRIPTION OF EMBODIMENTS

Synthesis

Exemplary compounds of the invention have been synthesized as follows:

1. Epoxidation of 1-undecene and 1-octadecene: The epoxidation reactions were based on the work of Beller et al,² which employs 13% sodium hypochlorite in the presence of a stoichiometric or a catalytic amount of bromide. As shown in Scheme 1, 56% and 57% yields, respectively, have been obtained.

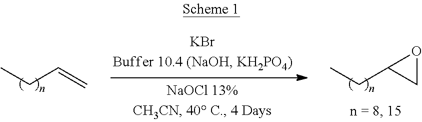

2. Regioselective ring-opening of 2-nonyl-oxirane (n=8) and 2-hexadecyl-oxirane (n=15) with aliphatic amines: The ring opening reactions, as depicted in Scheme 2, were carried out in the presences of the LiOTf catalyst to promote regioselective nucleophilic attack on the less hindered side of the epoxide ring, in acetonitrile based on the work of Auge et al.³ Series of aliphatic amines were used for the ring-opening reactions: diethyl-amine, butyl-amine, hexyl-amine, octyl-amine, dodecyl-amine, tetradecyl-amine and hexadecyl-amine. The yields ranged between 16-56%.

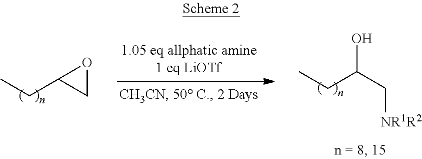

Characterization

LC-MS and ¹H-NMR spectroscopy were employed for the characterization of the compounds of the invention. LC-MS measurements have shown the expected mass and fragmentation pattern of each compound.

¹H-NMR: Varian VXR-300 MHz and 500 MHz, ¹³C-NMR and 2D-NMR experiments. As demonstrated in Scheme 3 below, the three distinct hydrogen atoms of the aminoalcohol backbone—H₁, H₂ and H₃—had distinct chemical shifts in their ¹H-NMR spectra. The chemical shift of H₁ appeared as a wide multiplet ranged between 3.60-4.00 ppm. Protons H₂/H′₂ appeared as a triplet ranged between 2.60-2.90 ppm and the protons H₃/H′₃ appeared as a quartet ranged between 2.80-3.00 ppm.

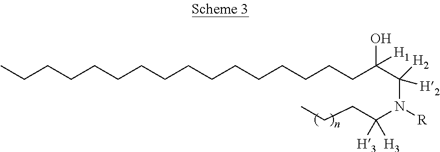

In order to confirm the regioselective ring-opening of the epoxide, 2D-NMR spectroscopy (COSY and NOESY experiments) was employed (results not shown).

General Procedure for the Epoxidation Reactions [Ref: 2]

To a stirred mixture of KBr (2.14 g, 18 mmol), buffer (60 ml, prepared by adjusting a 0.5M solution of KH₂PO₄ to a pH=10.4 with a 2M NaOH solution), 60 ml CH₃CN and the olefin (12 mmol) at 40° C., 28 ml of aqueous NaOCl 13% solution was added. The temperature and stirring were maintained for 4 days. Then, Na₂SO₃ (3 g, 24 mmol) was added and the mixture was extracted with ethyl acetate. The combined organic layers were dried over MgSO₄ and then the solvent was evaporated. The crude epoxide was purified by column chromatography (hexane/ethyl acetate 10:0.5).

### EXAMPLE 1

### 2-nonyl-oxirane (n=8 in the Oxirane of Scheme 2)

¹H-NMR (300 MHz, CDCl₃) δ (ppm): 0.875 (t, 3H), 1.265-1.576 (br s, m, 16H), 2.453 (dd, 1H), 2.600 (t, 1H), 2.894 (m, 1H). Yield 56%.

### EXAMPLE 2

### 2-hexadecyl-oxirane (n=15 in the Oxirane of Scheme 2)

¹H-NMR (300 MHz, CDCl₃) δ (ppm): 0.867 (t, 3H), 1.246-1.588 (br s, m, 30H), 2.455 (dd, 1H), 2.739 (t, 1H), 2.897 (m, 1H). Yield 57%.

General Procedure for the Epoxide Regioselective Ring-Opening [Ref: 3]

A solution of the epoxide prepared as above (0.58 mmol) in 3 ml anhydrous CH₃CN was treated with anhydrous LiOTf (1.0 eq, 90 mg, 0.58 mmol) under Ar. After stirring the mixture for 3 hr at 50° C., the aliphatic amine (1.05 eq, 147 mg, 0.61 mmol) was added, and the solution was allowed to react for 2 days. After the end of the reaction, a saturated solution of NH₄Cl (5 ml) was added and the adduct was extracted with hot ethyl acetate. The organic phase was washed with 2M HCl and then with water. The organic extracts were dried over MgSO₄ filtered off and then evaporated. The obtained amino-alcohols were recrystallized from ethyl acetate.

### Compound A1: 1-DEA-amino-octadecan-2-ol

¹H-NMR (300 MHz, CDCl₃) δ (ppm): 0.868 (t, 3H), 1.226 (br s, 29H), 1.349 (t, 6H), 2.923 (d, 2H), 1.455 (br s, 2H), 3.213 (q, 4H), 3.943 (m, 1H).

MS: 342.47 m/z. Yield 70%.

### Compound A2: 1-Butyl-amino-octadecan-2-ol

¹H-NMR (300 MHz, CDCl₃) δ (ppm): 0.870 (t, 3H), 0.953 (t, 3H), 1.243 (br s, 32H), 1.458 (br s 2H), 1.857 (p, 2H), 2.896 (t, 2H), 2.994 (q, 2H), 4.114 (m, 1H).

MS: 342.67 m/z. Yield 52%.

### Compound A3: 1-Hexyl-amino-octadecan-2-ol

¹H-NMR (300 MHz, CDCl₃) δ (ppm): 0.870 (t, 6H), 1.243(br s, 36H), 1.458 (br s, 2H), 1.838 (p, 2H), 2.865-2.973 (br m, 4H), 4.095 (m, 1H).

MS: 370.87 m/z. Yield 25%.

### Compound A4: 1-Octyl-amino-octadecan-2-ol

¹H-NMR (300 MHz, CDCl₃) δ (ppm): 0.878 (t, 6H), 1.253(br s, 40H), 1.468 (br s, 2H), 1.846 (p, 2H), 2.839-2.975 (br m, 4H), 4.084 (m, 1H).

MS: 398.40 m/z. Yield 20%.

### Compound A5: 1-Dodecyl-amino-octadecan-2-ol

¹H-NMR (300 MHz, CDCl₃) δ (ppm): 0.873 (t, 6H), 1.246(br s, 48H), 1.463 (br s, 2H), 1.831 (p, 2H), 2.854-2.973 (br m, 4H), 4.092 (m, 1H).

MS: 419.80 m/z. Yield 35%.

### Compound A6: DEA-amino-undecan-2-ol

¹H-NMR (300 MHz, CDCl₃) δ (ppm): 0.850 (t, 3H), 1.226(br s, 15H), 1.349 (t, 6H), 1.455 (br s, 2H), 3.012 (d, 2H), 3.350 (q, 4H), 3.943 (m, 1H).

### Compound A7: 1-Butyl-amino-undecan-2-ol

¹H-NMR (300 MHz, CDCl₃) δ (ppm): 0.850 (t, 3H), 0.910 (t, 3H), 1.226 (br s, 18H), 1.434 (br s, 2H), 1.672 (p, 2H), 2.920-3.053 (m, 4H), 3.913 (m, 1H).

### Compound A8: 1-Hexyl-amino-undecan-2-ol

¹H-NMR (300 MHz, CDCl₃) δ (ppm): 0.863 (t, 6H), 1.244 (br s, 22H), 1.464 (br s, 2H), 1.713 (p, 2H), 2.908-3.026 (br m, 4H), 3.966 (m, 1H).

MS: 272.40 m/z. Yield 56%.

### Compound A9: 1-Octyl-amino-undecan-2-ol

¹H-NMR (300 MHz, CDCl₃) δ (ppm): 0.879 (t, 6H), 1.258(br s, 26H), 1.464 (br s, 2H), 1.778 (p, 2H), 2.955-3.026 (br m, 4H), 4.013 (m, 1H).

MS: 300.40 m/z. Yield 23%.

### Compound A10: 1-Dodecyl-amino-undecan-2-ol

¹H-NMR (300 MHz, CDCl₃) δ (ppm): 0.880 (t, 6H), 1.252(br s, 36H), 1.473 (br s, 2H), 1.858 (p, 2H), 2.879-2.969 (br m, 4H), 4.106 (m, 1H).

MS: 356.47 m/z. Yield 30%.

### Compound A11: 1-tetradecyl-amino-undecan-2-ol

¹H-NMR (300 MHz, CDCl₃) δ (ppm): 0.886 (t, 6H), 1.258(br s, 38H), 1.465 (br s, 2H), 1.872 (p, 2H), 2.829-3.057 (br m, 4H), 4.120 (m, 1H).

MS: 384.42 m/z. Yield 30%.

### Compound A12: 1-hexadecyl-amino-undecan-2-ol

¹H-NMR (300 MHz, CDCl₃) δ (ppm): 0.886 (t, 6H), 1.260 (br s, 42H), 1.479 (br s, 2H), 1.772 (p, 2H), 2.880-3.243 (br m, 4H), 4.017 (m, 1H).

MS: 412.45 m/z. Yield 50%.

General Procedure for the Preparation of Arylated Derivatives [Ref: 5]

As shown in Scheme 4, the arylated derivatives may be prepared from an arylated vinyl.

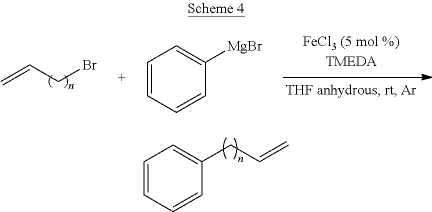

To a mixture of the desired alkenyl halide (50 mmol) and 0.1M FeCl₃ (25 ml in THF) under Ar, a mixture of PhMgBr (72 ml of a 0.93M THF Solution, 67 mmol) and TMEDA (7.78 g, 67 mmol) is added via syringe pump at a rate of 1.0 ml/min at rt. The reaction mixture is stirred for additional 10 min. Then, a saturated aqueous solution of NH₄Cl (25 ml) is added to quench the reaction and the mixture is extracted several times with CH₂Cl₂, and washed with H₂O. The combined organic layer is dried over MgSO₄ and the solvent was evaporated. The crude olefin is purified by column chromatography.

The epoxidation and the ring-opening reactions of the arylated precursors are carried out according to procedures described above.

The variety of alkenyl halides which may be employed in the preparation of arylated derivatives of formula (I) is depicted in Annex A.

General Procedure for the Solutol Emulsions Preparation

The amino-alcohol compound of the invention (4 μmol) was mixed with Solutol HS15 (6.0 mg) and heated to 70-80° C. Hot water (1 ml) was added to the mixture and stirred thoroughly. Thickening occurs initially due to hydration and reaches a maximum when half of the water has been added. The viscosity decreases as more water is added.

Solubility

The solubility of compounds of the invention in ethanol and DMSO was tested in order to study their cytotoxicity. 20 mM stock solutions have been prepared and are listed in Table 1. As may be noted from Table 1, A1 has showed good solubility in both solvents while A2 was only soluble in DMSO. Moreover, compounds A8-A12 showed good solubility in ethanol at slightly higher temperatures. Compounds A3-A5 exhibited poor solubility, thus lipidic emulsions using the Solutol-HS15 reagent have been prepared.

TABLE 1
Solubility of compounds of the invention: ✓-soluble; X-poor
solubility; * need slight heating to dissolve.

| | DMSO | Ethanol | Solutol-HS15 |
|---|---|---|---|
| A1 | ✓ | ✓ | ✓ |
| A2 | ✓* | X | ✓ |
| A3 | X | X | ✓ |
| A4 | X | X | ✓ |
| A5 | X | X | ✓ |
| A8 | ✓ | ✓ | |
| A9 | X | ✓* | |
| A10 | X | ✓* | |
| A11 | X | ✓* | |
| A12 | X | ✓* | |

Biological Tests

Cytotoxicity Tests (IC₅₀ Determination by MTT test)

The cytotoxicity of the compounds of the invention A1-A12 was tested in comparison to cis-Pt in four cancer cell-lines:

A2780 (ovarian carcinoma cell line),

A2780cisR (ovarian carcinoma-cisPt resistance cell line),

CRL-5803 (non-small cell lung carcinoma cell line), and

HT-29 (Human colon adenocarcinoma grade II) by MTT test after an incubation of 24 hours.

Culture Medium Preparation for Attached Cell-lines: A2780, A2780cisR, CRL-5803, HT-29.
Medium RPMI-1640: 86%
Serum (FCS-fatal calf serum/FBS-fatal bovine serum): 10%
L-Glutamine: 1%
Antibiotics-penicillin-streptomycin(P/S): 1%
Na pyruvate: 1%
HEPES: 1%

Enzymatic Assay in Cells (SMS, GCS and CDase Enzyme inhibition Studies)
Plate 10,000 cells in 0.5 ml culture media per well in a 48 well plate.
Incubate (37° C., 5% CO₂) overnight to allow the cells to attach to the wells.
Add 10 μl of the fluorescent reagent BodiPy-12-Cer 0.5 μM/well.
After 48 hr incubation with the BodiPy-12-Cer, add 10 μl of the drug of interest dissolved in DMSO/Ethanol/solutol to each well.
Incubate (37° C., 5% CO₂) for another 24 hr.
Remove the media.
Wash each well with 200 μl PBS.
Add 2000 trypsin allow sitting for 5 minutes in incubator.
Neutralize Trypsin with culture medium (complete to 1 ml)
Add suspension to 1.5 ml ephendorf.
Centrifuge for 5 minutes at 1200 rpm then remove the media.
Extract the cells with 1 ml of 1:2 CH₂Cl₂:MeOH by strong vortex stirring followed by centrifuge for 5 minutes at 13400 rpm. Then add the extraction into 2 ml ephendorf.
Extract the cells again with the same procedure with 1 ml of 1:1 CH₂Cl₂:MeOH.
Remove the solvent.
Add 2000 ethanol into the 2 ml ephendorf, do strong vortex stirring followed by centrifuge for 5 minutes at 13400 rpm.
Take 150 μl of the above sample and inject to the HPLC to quantify the relative metabolite relatively to control samples.
HPLC method conditions are:

| Time | Flow | | H₂O | |
|---|---|---|---|---|
| (min) | (ml/min) | Methanol % | (0.1% TFA) | CH₂Cl₂ % |
| 0 | 1.5 | 80 | 20 | 0 |
| 3 | 1.5 | 80 | 20 | 0 |
| 4 | 1.5 | 90 | 10 | 0 |
| 8 | 1.5 | 60 | 0 | 40 |

Conditions: RP-8 Column, EX. _{λ} 505 nm, EM. _{λ} 530 nm

Apoptosis Determination by Caspase-3 Assay

The assay was done according to the protocol of EnzCheck Caspase-3 Assay Kit #2 (Invitrogen) [Ref: 4].

TABLE 2
Cytotoxicity test for Compounds A1-A5: IC₅₀ (μM) ± SD (MTT-Test),
24 hr Incubation, 20000 cells/well, 10% Serum, solutol solutions.

| Compound | | A2780 | A2780cisR | HT-29 | CRL5803 |
|---|---|---|---|---|---|
| A1 | C18-DEA | 17 ± 4 | 21 ± 1 | 24 ± 4 | 40 ± 3 |
| A2 | C18-ButylA | 11 ± 3 | 12 ± 3 | 16 ± 2 | 32 ± 4 |
| A3 | C18-HexylA | 11 ± 1 | 9 ± 1 | 19 ± 5 | 18 ± 2 |
| A4 | C18-OctylA | 14 ± 3 | 12 ± 2 | 15 ± 6 | 29 ± 4 |
| A5 | C18-DodecylA | >100 | >100 | >100 | >100 |

| | | | | |
|---|---|---|---|---|
| cis-Pt | 30 ± 1 | >100 | >100 | >100 |

TABLE 3
Cytotoxicity test for Compounds A8-A12: IC₅₀ (μM) ± SD (MTT-Test),
24 hr Incubation, 20000 cells/well, 10% Serum, ethanol solutions.

| Compound | | A2780 | A2780cisR | HT-29 | CRL5803 |
|---|---|---|---|---|---|
| A8 | C11-HexylA | 16 ± 1 | 22 ± 3 | 33 ± 4 | 33 ± 6 |
| A9 | C11-OctylA | 7 ± 1 | 10 ± 1 | 11 ± 2 | 14 ± 1 |
| A10 | C11-DodecylA | 4 ± 1 | 4 ± 1 | 8 ± 2 | 10 ± 2 |
| A11 | C11-TetradecylA | 9 ± 2 | 9 ± 3 | 30 ± 7 | 33 ± 6 |
| A12 | C11-HexadecylA | >100 | >100 | >100 | >100 |

| | | | | |
|---|---|---|---|---|
| cis-Pt | 30 ± 1 | >100 | >100 | >100 |

As may be noted from Tables 2 and 3, compounds A1-A4 and A8-A11 exhibited anticancer activity as compared to cis-Pt. Compounds A9 and A10 were determined to be most active. In addition, the activity of the compounds showed to be approximately the same for the ovarian and its cis-Pt resistant cell lines.

Lipophilicity and IC₅₀

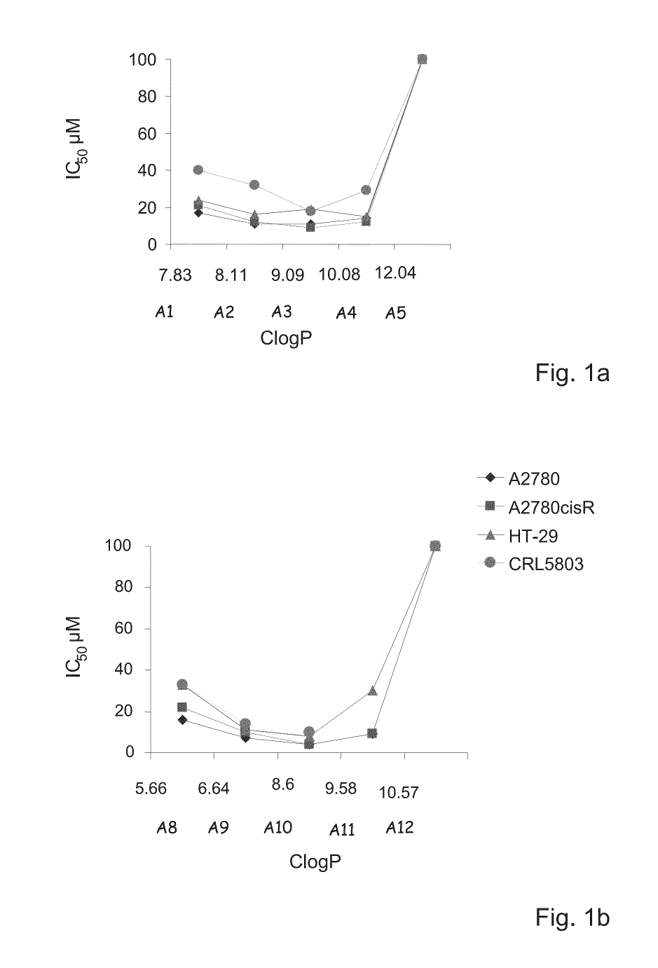

The correlation between the cytotoxicity of the compounds and their lipophilicity or, to put it differently, the correlation between the chain length on the different sites of the molecule and the reactivity was also tested. The IC₅₀ values versus the calculated log P of the compounds of the invention are plotted in FIGS. 1A and 1B. As may be noted, for compounds A1-A5 the optimal lipophilicity range was 9-10 while for compounds A8-A12 the optimal one was 8-9.

Enzymatic Examinations in Cells

A fluorescent procedure was utilized in order to study the enzymatic inhibition of glucosylceramide synthase (GCS), ceramidases (CDase) and sphingomyelin synthase (SMS) enzymes, which can shed some light on a possible mechanism for ceramide accumulation and apoptosis. The fluorescent procedure was based on a 48-hr incubation of cancer cells with the fluorescent substrate, Bodipy (4,4-difluoro-4-bora-3a,4a-diaza-s-indacene), conjugated ceramide (Bodipy-12-CER) followed by another 24-hr period of incubation in the presence of nontoxic concentrations of compounds of the invention (72 hr incubation in total). After a predetermine incubation period, cells were washed, their lipids were extracted and the various lipids containing fluorescent fatty acid-ceramide, SPM and GC were quantified by HPLC.

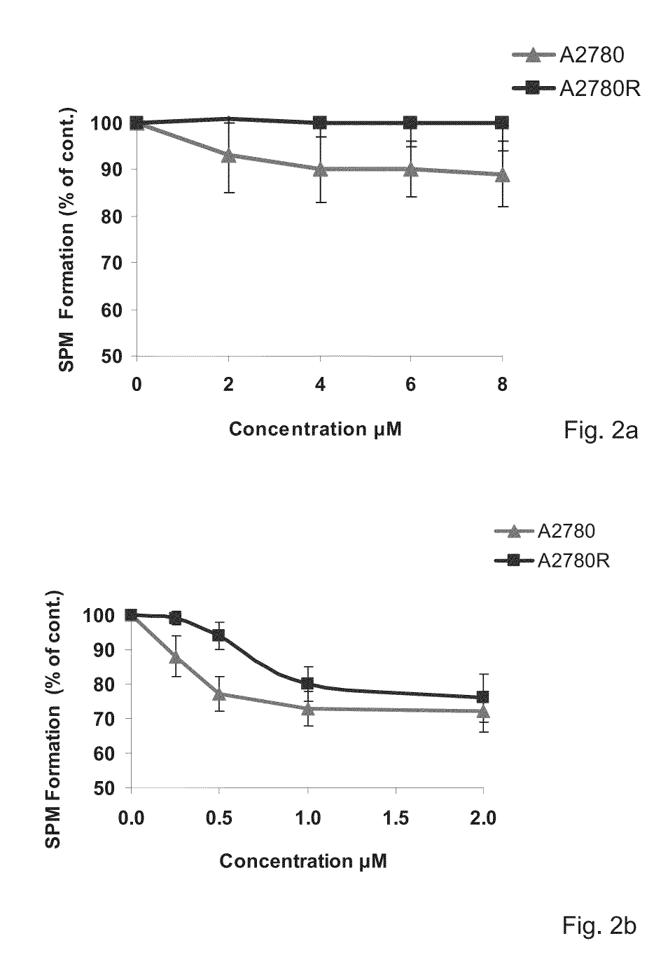

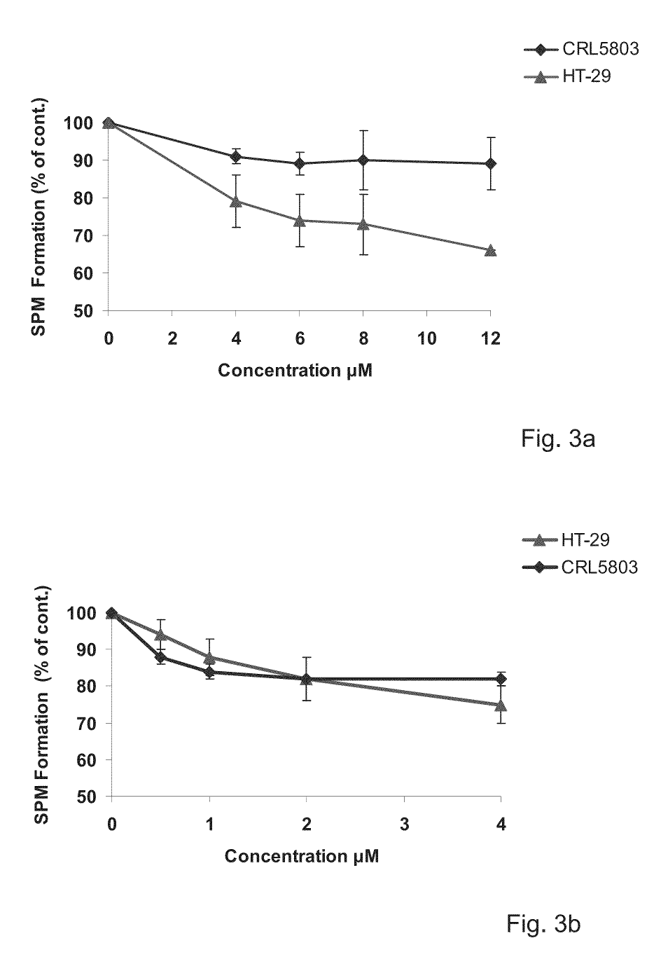

Herein the results of the effect of A2 (moderate activity) and A10 (highest activity) compounds on the formation of the fluorescent metabolites after 72 hr incubation within four cancer cell-lines: A2780, A2780cisR (FIGS. 2a and 2b), HT-29 and CRL5803 (FIGS. 3a and 3b) showed a reduction in SPM formation related to the control. For example: A2 inhibited the SPM formation at 6 μM by ˜10% in A2780 cells but had no significant effect on A2780cisR cell (FIG. 2a).While, A10 had about 22% and 8% effect in A2780 and A2780cisR relatively at 0.5 μM (FIG. 2b). This reduction may be as a result of an enzymatic inhibition of sphingomyelin synthase (SMS) enzyme, which lead to ceramide accumulation and apoptosis.

Apoptosis Determination by Caspase-3 Assay

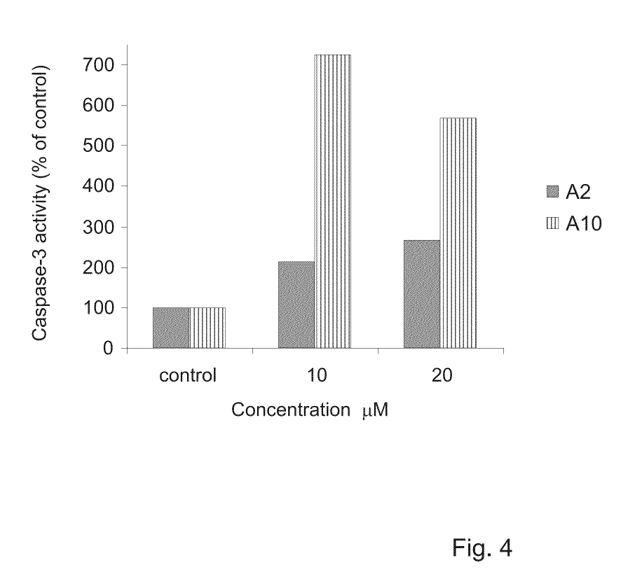

Caspase-3 was checked using invitrogene protocol. 8 hr incubation was done. A 7-fold increase was found at 10 μM for compound A10 and 3 fold increase for 20 μM of compound A2, as shown in FIG. 4.

### Annex A

Alkenyl halides selected from:

Bronimated alkenyls:

1. 1-bromoethylene; CAS-593-60-2

2. 3-bromo-1-propene; CAS-106-95-6

3. 4-bromo-1-butene; CAS-5162-44-7

4. 6-bromo-1-hexene; CAS-2695-47-8

5. 7-bromo-1-heptene; CAS-4117-09-3

6. 8-bromo-1-octene; CAS-2695-48-9

7. 10-bromo-1-decene; CAS-62871-09-4

Chlorinated alkenyls:

1. 1-chloroethylene; CAS-75-01-4

2. 3-chloro-1-propene; CAS-107-05-1

3. 6-chloro-1-hexene; CAS-928-89-2

4. 11-chloro-1-undecene; CAS-872-17-3

Iodinated alkenyls:

1. 3-iodo-1-propene; CAS-556-56-9

Aryl Grignard reagents:

1. phenylmagnesium bromide; CAS-100-58-3

2. bromo(4-methylphenyl)magnesium bromide; CAS-4294-57-9

3. bromo(4-ethylphenyl)magnesium bromide; CAS-22873-28-5

4. bromo(4-methoxyphenyl)magnesium bromide; CAS-13139-86-1

5. bromo(4-chlorophenyl)magnesium bromide; CAS-873-77-8

6. bromo(4-isopropylphenyl)magnesium bromide; CAS-635669

7. bromo[4-(diethylamino)phenyl]magnesium bromide; CAS-7353-91-5

8. 1,3-benzodioxol-5-yl(bromo)magnesium bromide; CAS-17680-04-5

9. bromo[4-(methylsulfanyl)phenyl]magnesium bromide; CAS-18620-04-7

10. bromo(2-naphthyl)magnesium bromide; CAS-21473-01-8

11. [1,1′-biphenyl]-2-yl magnesium bromide; CAS-82214-69-5

Arylated halides:

1. (2-bromoethyl)benzene; CAS-103-63-9

2. 1-(2-bromoethyl)-4-methylbenzene; CAS-6529-51-7

3. 4-(2-bromoethyl)phenol; CAS-14140-15-9

4. (3-bromopropyl)benzene; CAS-637-59-2

5. 1,3-dibromo-5-p-tolyl-adamantane; CAS-

6. (2-chloroethyl)benzene; CAS-622-24-2

7. (2-chloro-1,1-dimethyulethyl)benzene; CAS-515-40-2

8. (3-chloropropyl)benzne; CAS-104-52-9

9. (4-chlorobutyl)benzene; CAS-4830-93-7

The Catalyst:

Iron(III) chloride FeCl₃; CAS-7705-08-0

The Additive:

N,N,N′,N′-tetramethylethylenediamine (TMEDA); CAS-110-18-9

1. A compound of the general formula (I), or a salt or isomer thereof: 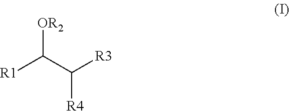
wherein
R₁ is selected from C₈-C₁₄ alkyl, C₁₆-C₂₄ alkyl, C₂-C₂₄ alkenyl, and C₂-C₂₄ alkynyl, each being optionally substituted with at least one substituent selected from —OH, —OR₅ and optionally substituted C₆-C₁₀ aryl;
R₂ is selected from —H, C₁-C₆ alkyl, C₂-C₆ alkenyl, C₂-C₆ alkynyl, and —C(═O)—R₆;
R₃ is selected from —H, C₁-C₂₄ alkyl, C₂-C₂₄ alkenyl, and C₂-C₂₄ alkynyl, each being optionally substituted with at least one substituent selected from —OH, —OR₇ and optionally substituted C₆-C₁₀ aryl;
R₄ is selected from —NHR₈, —NR₈R₉ and —N⁺R₈R₉R₁₀;
each of R₅ and R₇, independently of each other is selected from C₁-C₆ alkyl, C₂-C₆ alkenyl, C₂-C₆ alkynyl, and —C(═O)—R₁₁;
each of R₆ and R₁₁, independently of each other is selected from C₁-C₆ alkyl, C₂-C₆ alkenyl, and C₂-C₆ alkynyl;
each of R₈ and R₉, independently of the other, is selected from C₁-C₂₄ alkyl, C₂-C₂₄ alkenyl, C₂-C₂₄ alkynyl, —C(═S)—R₁₂, —C(═S)—NR₁₂R₁₃, —SO₂—R₁₂, —C(═O)—R₁₂, and —C(═O)—NR₁₂R₁₃;
R₁₀ is selected from —H, C₁-C₂₄ alkyl, C₂-C₂₄ alkenyl, C₂-C₂₄ alkynyl, —C(═S)—R₁₂, —C(═S)—NR₁₂R₁₃, —SO₂—R₁₂, —C(═O)—R₁₂, and —C(═O)—NR₁₂R₁₃;
when R₄ is —NR₈R₉, R₈ and R₉, together with the N atom to which they are bonded may form a heterocyclic group, optionally comprising one or more additional atom selected from N, S, and O;
each of R₁₂ and R₁₃, independently of each other is selected from C₁-C₆ alkyl, C₂-C₆ alkenyl and C₂-C₆ alkynyl; and
wherein at least one of R₁ and R₈ is selected from C₉-C₂₄ alkyl, C₉-C₂₄ alkenyl and C₉-C₂₄ alkynyl.

2. The compound according to claim 1, R₁ is selected from unsubstituted C₈-C₁₄ alkyl, and C₁₆-C₂₄ alkyl.

3. The compound according to claim 2, wherein R₁ is an alkyl having 9, 10, 11, 12, 13, 14, 16, 17, 18, 19, 20, 21, 22, 23 or 24 carbon atoms in a continuous aliphatic chain.

4. The compound according to claim 3 being a compound of formula (II): 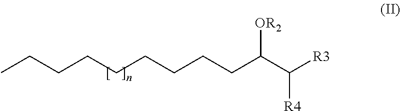
wherein
n is an integer selected from 1, 2, 3, 4, 5, 6, 8, 9, 10, 11, 12, 13, 14, 15, and 16, and each of R₂, R₃ and R₄ are as defined in claim 1.

5. The compound according to claim 1, being a compound of the general formula (III): 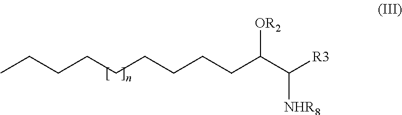
wherein
n, R₂ and R₃ are as defined in claim 1 and R₈ is different from —H.

6. The compound according to claim 1, wherein R₄ is —NR₈R₉, the compound being of formula (IV): 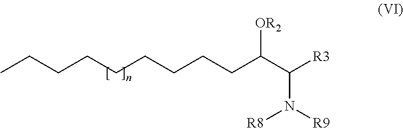
wherein
each of n, R₂ and R₃ are as defined in claim 1 and R₈ and R₉ are each different from —H.

7. The compound according to claim 6, wherein each of R₈ and R₉ is different or same —C₁-C₂₄ alkyl.

8. A compound selected from compounds herein designated A1, A2, A3, A4, A5, A6, A7, A8, A9, A10, A11 and A12.

9. A pharmaceutical composition comprising at least one compound according to claim 8.

10. A pharmaceutical composition comprising at least one compound according to claim 1.

11. A method for the treatment of a disease or disorder, said method comprising administering to a subject suffering from said disease or disorder an effective amount of at least one compound according to claim 1.

12. The method according to claim 11, wherein said disease or disorder is cancer.

13. The method according to claim 12, wherein said cancer is a carcinoma, a sarcoma, a liquid tumor, a lymphoma, leukemia, and solid tumor.

14. The method according to claim 13, wherein said cancer is selected from colon, lung, breast, pancreas, prostate and ovarian cancers.

15. The method according to claim 13, wherein said cancer is selected from colon, lung, and ovarian cancers.

16. The method according to claim 11, wherein said disease is a Lipid Storage Disease.

17. The method according to claim 16, wherein the disease is Niemann-Pick.

18. A method for the treatment of a disease or disorder, said method comprising administering to a subject suffering from said disease or disorder an effective amount of at least one compound according to claim 8.

19. The method according to claim 18, wherein said disease or disorder is cancer.

20. The method according to claim 19, wherein said cancer is a carcinoma, a sarcoma, a liquid tumor, a lymphoma, leukemia, and solid tumor.

21. The method according to claim 20, wherein said cancer is selected from colon, lung, breast, pancreas, prostate and ovarian cancers.

22. The method according to claim 18, wherein said disease is a Lipid Storage Disease.

23. The method according to claim 22, wherein the disease is Niemann-Pick.

24. A method of inducing cancer cell death, in vivo or in vitro, said method comprising contacting said cells with at least one compound according to claim 1.

25. A method of inducing cancer cell death, in vivo or in vitro, said method comprising contacting said cells with at least one compound according to claim 8.

26. A compound selected from:

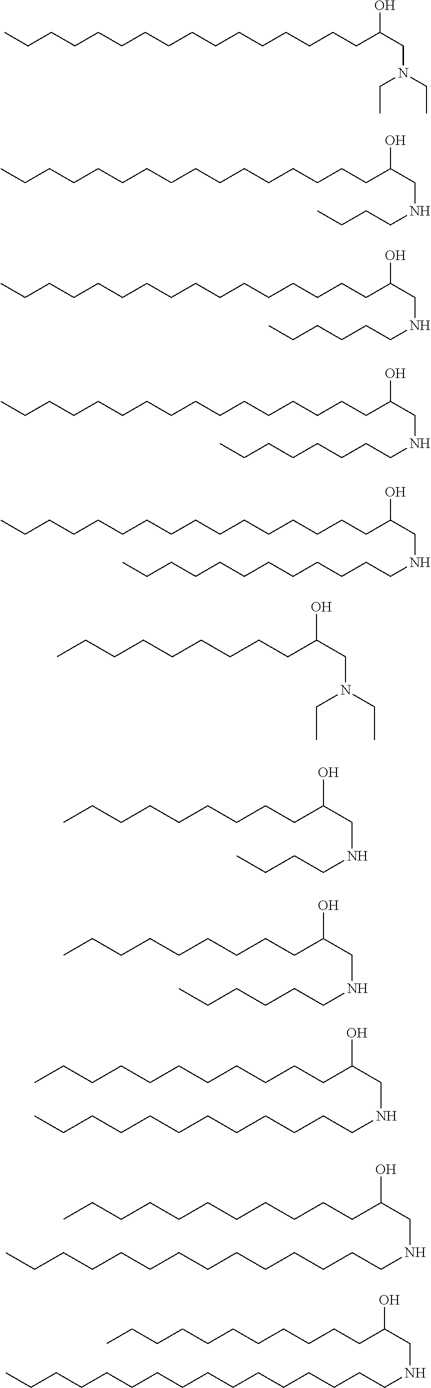

27. A compound selected from:
1-Dodecyl-amino-dodecan-2-ol,
1-Decyl-amino-dodecan-2-ol,
1-DEA-amino-octadecan-2-ol,
1-Butyl-amino-octadecan-2-ol,
1-Hexyl-amino-octadecan-2-ol,
1-Octyl-amino-octadecan-2-ol,
1-Dodecyl-amino-octadecan-2-ol,
1-DEA-amino-undecan-2-ol,
1-Butyl-amino-undecan-2-ol,
1-Hexyl-amino-undecan-2-ol,
1-Dodecyl-amino-tridecan-2-ol,
1-tetradecyl-amino-tridecan-2-ol, and
1-hexadecyl-amino-tridecan-2-ol.

28. A compound of claim 27 selected from:
1-Dodecyl-amino-dodecan-2-ol and,
1-Decyl-amino-dodecan-2-ol.
